(19) United States
(12) Patent Application Publication
(10) Pub. No.: US 2014/0243354 A1
(43) Pub. Date: Aug. 28, 2014

(54) COMBINATIONS OF INDIGO NATURALIS AND BERBERINE AND USES THEREOF

(71) Applicant: GALDERMA RESEARCH & DEVELOPMENT SNC, (US)

(72) Inventors: Laurent CHANTALAT, Antibes (FR); Philippe ANDRES, Peymeinade (FR)

(73) Assignee: GALDERMA RESEARCH & DEVELOPMENT SNC, Biot (FR)

(21) Appl. No.: 13/774,413

(22) Filed: Feb. 22, 2013

(51) Int. Cl. A61K 31/4375; A61K 31/136; A61K 31/519; A61K 31/4015; A61K 31/404

(52) U.S. Cl. CPC A61K 31/4375; A61K 31/4015; A61K 31/404; A61K 31/519; A61K 31/136
USPC 514/257; 514/280

(57) ABSTRACT

Methods and composition for treating or preventing an inflammatory dermatological disease or condition, such as psoriasis, in a subject are described. The methods involve topical applications to the subject a therapeutically effective amount of a composition comprising at least one indigo naturalis composition, beberine and a pharmaceutically acceptable carrier.

### BACKGROUND OF THE INVENTION

Some dermatological conditions can be painful, irritating, stressful and difficult to treat. For example, psoriasis is a chronic inflammatory skin disease that may appear on the skin at any area of the body, in particular head, limbs, elbows, knees, etc. Red papules or plaques having clear borders are frequently observed on the skins of psoriasis patients, and the surfaces of the skin lesions of the patients may be covered by silvery-white scales. Some psoriasis patients may even develop arthritis or other symptoms at the same time.

According to skin symptoms, psoriasis is clinically classified into the following four types: chronic plaque psoriasis, guttate psoriasis, erythrodermic psoriasis, and pustular psoriasis. Chronic plaque psoriasis, also known as vulgaris psoriasis, is most commonly seen amongst all the types of psoriasis, and around 80%-90% of psoriasis patients belong to this type. Guttate psoriasis is characterized by numerous small pink or red teardrop-shaped lesions on patients' skins and usually occurs in children from age 7 to age 10, in which most of the patients are found to have suffered a streptococcal infection one or two weeks before the onset of the disease. Erythrodermic psoriasis is characterized by the appearance of erythema or exfoliation over the whole body's surface or more than 90% of the body's surface, and under more severe conditions, the occurrence of itching over the whole body, which may be further accompanied by fever and malaise. Moreover, generalized acute pustular psoriasis may even be induced in patients with erythrodermic psoriasis. Pustular psoriasis can be further categorized into generalized pustular psoriasis and localized pustular psoriasis (e.g., pustulosis palmaris et plantaris and acrodermatitis perstans). Besides, around 10% of the psoriasis patients have a complication of psoriatic arthritis, which is a CD8 T cell-driven autoimmune inflammatory disorder that affects ligaments, tendons, fascia, and spinal or peripheral joints, while up to 90% of the patients have suffered nail psoriasis.

Due to the complicated pathological conditions and symptoms involved therein, the pathogenesis of psoriasis has yet to be well understood to date. Some studies have revealed that inheritance plays an important role in the pathogenesis of psoriasis, and trauma, infection, stress, endocrine factors, metabolism factors, weather and medication may induce or aggravate psoriasis. After the onset of psoriasis, this disease may recur repeatedly over patients' lifetime and no eradication of the same is possible.

The conventional treatments for psoriasis are generally designed according to the age, gender, occupation and cognitive ability of the patient, the types and distribution of lesions, patient's response(s) to previous therapeutic method(s), and other medical histories of the patient. The primary therapeutic methods for psoriasis include topical therapy, systemic therapy, injection of biologics and phototherapy. Compositions for topical therapy include, e.g., corticosteroids, anthralin (available as Margiton®), coal tar (available as Polytar®), calcitriol (available as Silkis®), tazarotene (available as Tazorac®), salicylic acid, etc., and these compositions are suitable for treating psoriasis patients with mild symptoms.

Oral preparations of methotrexate (MTX), cyclosporine, retinoids, etc., are commonly used for systemic therapy and are suitable for treating psoriasis patients with medium to severe symptoms. Biologics include alefacept (available as Amevive®), efalizumab (available as Raptiva etanercept (available as Enbrel®) and adalimumab (available as Humire), and they are suited for injecting into psoriasis patients with medium to severe symptoms. Phototherapy, e.g., ultraviolet B (UVB) phototherapy, photochemotherapy such as psoralen plus ultraviolet A (PUVA), etc., is suitable for treating psoriasis patients with severe symptoms.

However, long-term use of the conventional treatments described above may result in serious side effects or drug tolerance, thereby further reducing patient compliance. In view of the foregoing, many researchers have attempted to explore, from traditional Chinese medicines (TCM), active components that can be used to treat psoriasis.

Chinese herbs have been used in the treatment of psoriasis. See, e.g., Tse, Clin Exp Dermatol 2003, 28:469-75; and Tan et al., Chin J. Integr Med., 2011, 17: 150-3. Tablets of indigo naturalis had been shown to be effective in treating psoriasis, but with gastrointestinal side effects. Yuan et al., J. Tradit Chin Med, 1982, 2:306. Successful treatment of pediatric psoriasis was reported with an ointment containing indigo naturalis (Qing Dai), Scutellaria baicalensis-Georgi (Huang Qin) and Cortex phellodendri (Huang Bai). Lin et al., Pediatr Dermatol 2006, 23: 507-10. A new Pulian ointment containing Radix scutellariae, Cortex phellodendri, indigo naturalis and Radix arnebiae seu lithospermi was also shown to be effective in treating psoriasis of blood-heat syndrome. Zhou et al., Chin J. Integr Med., 2009, 15: 409-14.

Indigo naturalis (also referred to as natural indigo; Chinese: Quing dai) is a dark-blue plant pigment extracted from leaves of an indigo-producing plant such as Baphicacanthus cusia (Nees) Bremek., Polygonum tinctorium Lour., Isatis indigotica Fort., etc.

Indigo naturalis has been clinically used to treat, psoriasis. See Y. K. Lin et al. (2006a), Pediatric Dermatology, 23(5):507-510; Y. K. Lin et al. (2006b), Clinical and Experimental Dermatology, 99-100; and Y. K. Lin et al. (2007), Dermatology, 214:155-161; Y. K. Lin et al. (2008), Arch Dermatology, 144(11):1457-1464. Indigo naturalis has been shown for its anti-psoriatic effect of indigo naturalis on the proliferation and differentiation of keratinocytes. See Y. K. Lin et al. (2009a), Journal of Dermatological Science, 54:168-174.

It has been known that various active components can be isolated from indigo naturalis prepared from different indigo-producing plants, amongst which the most common active components are indigo, indirubin, tryptanthrin, etc. It has been reported that indirubin has anti-tumor, anti-inflammation, and immunomodulatory effects and has been considered to have potential in the treatment of chronic myelocytic leukemia (G. Eisenbrand et al. (2000), J. Cancer Res. Clin. Oncol., 130:627-635; T. Kunikata et al. (2000), European Journal of Pharmacology, 410:93-100; N. K. Mak et al. (2004), Biochemical Pharmacology, 67:167-174; S. Leclerc et al. (2001), The Journal of biological Chemistry, 276:251-260).

The anti-inflammatory effects of indigo naturalis, indigo, indirubin, and tryptanthrin in human neutrophils have been investigated and compared. It was found that only the indigo naturalis sample inhibited human neutrophil proinflammatory responses, including respiratory burst and degranulation, which were mediated through blocking MAPK and Ca2+ signaling pathways. The results supported the concept of developing a whole herbal extract of indigo naturalis, rather than the major component(s) contained therein, for treating neutrophilic inflammation (Y. K Lin et al. (2009b), Journal of Ethnopharmacology, 125:51-58).

However, the indigo naturalis ointments often have the drawbacks of unpleasant odor and deep blue color that may leave stains on nails, skins and clothes, thereby affecting patients' compliance.

There remains a need of novel effective and safe methods and compositions for treating or preventing a dermatological condition, such as psoriasis and related symptoms. Such methods and compositions are described in the present application.

### BRIEF SUMMARY OF THE INVENTION

A synergistic effect is observed from a combination comprising berberine and at least one indigo naturalis composition for treating or preventing a dermatological condition, such as psoriasis, with no or little side effects.

Therefore, in one general aspect, embodiments of the present invention relate to a topical composition for treating an inflammatory dermatological condition, comprising: a) berberine; b) at least one indigo naturalis composition; and c) a topically acceptable carrier, wherein a synergistic effect is obtained between the berberine and the at least one indigo naturalis composition.

The new compositions of the present invention reduce the blue discoloration of nails, skins and clothes, thus more aesthetically pleasing for topical application, and yet, remain effective for the treatment of psoriasis. Moreover, the new compositions cause less skin irritation, thus making the product more user-friendly.

In another general aspect, embodiments of the present invention relate to a method of treating or preventing an inflammatory dermatological condition in a subject. The method comprises topically administering to a skin area of the subject a therapeutically effective amount of a topical composition comprising: a) berberine; b) at least one indigo naturalis composition; and c) a topically acceptable carrier, wherein a synergistic effect is obtained between the berberine and the at least one indigo naturalis composition.

In a preferred embodiment, the inflammatory dermatological condition is psoriasis or a symptom associated therewith.

Other aspects, features and advantages of the invention will be apparent from the following disclosure, including the detailed description of the invention and its preferred embodiments and the appended claims.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

The term “topically administrable composition,” a “topical composition,” or a “topical formulation,” as used herein, means any formulation or composition which is pharmaceutically and/or cosmetically acceptable for topical delivery of the specified compounds according to embodiments of the invention. Exemplary forms of formulation that can be used for topical administration in embodiments of the present invention include, but are not limited to, sprays, mists, aerosols, solutions, lotions, gels, creams, ointments, pastes, unguents, emulsions, and suspensions. The choice of topically administrable composition will depend on several factors, including the nature of the symptoms to be treated or prevented, the physiochemical characteristics of the particular compound to be administered and of other excipients present, their stability in the formulation, available manufacturing equipment, and cost constraints.

As used herein, the term “composition” is intended to encompass a product comprising the specified ingredient in the specified amount, as well as any product which results, directly or indirectly, from combinations of the specified ingredient in the specified amount.

As used herein, the term “subject” means any animal, preferably a mammal, most preferably a human, to whom will be or has been administered compounds or topical formulations according to embodiments of the invention. The term “mammal” as used herein, encompasses any mammal. Examples of mammals include, but are not limited to, cows, horses, sheep, pigs, cats, dogs, mice, rats, rabbits, guinea pigs, monkeys, humans etc., more preferably, a human. Preferably, a subject is in need of, or has been the object of observation or experiment of, treatment or prevention of psoriasis and symptoms associated therewith.

In one embodiment, “treatment” or “treating” refers to an amelioration, prophylaxis, or reversal of a disease or disorder, or at least one discernible symptom thereof, for example, treating psoriasis by lessening the redness and/or scaly patches on the skin. In another embodiment, “treatment” or “treating” refers to an amelioration, prophylaxis, or reversal of at least one measurable physical parameter related to the disease or disorder being treated, not necessarily discernible in or by the mammal. In yet another embodiment, “treatment” or “treating” refers to inhibiting or slowing the progression of a disease or disorder, either physically, e.g., stabilization of a discernible symptom, physiologically, stabilization of a physical parameter, or both. In yet another embodiment, “treatment” or “treating” refers to delaying the onset of a disease or disorder.

In certain embodiments, compounds of interest are administered as a preventative measure. As used herein, “prevention” or “preventing” refers to a reduction of the risk of acquiring a given disease or disorder. In a preferred mode of the embodiment, the specified compounds are administered as a preventative measure to a subject having a predisposition to psoriasis, an inherent disease, even though symptoms of psoriasis are absent or minimal.

As used herein, a “therapeutically effective amount” means the amount of the composition that elicits the biological or medicinal response in a tissue system, animal or human that is being sought by a researcher, veterinarian, medical doctor or other clinician, which includes alleviation of the symptoms of the disease or disorder being treated. In a preferred embodiment, the therapeutically effective amount of a topical composition is effective to treat, improve the treatment of, or prophylactically prevent a dermatological condition, such as psoriasis or a symptom associated therewith.

As used herein, a “dermatological condition” is intended to encompass any type of dermatological condition that is in need of treatment or prevention, including acne, inflammatory skin conditions, non-inflammatory skin conditions, skin infections, UV-induced skin damage, skin cancer, alopecia, vitiligo, and wounds. Examples of inflammatory skin conditions or dermatological conditions include, but are not limited to, psoriasis, rosacea, eczema, and dermatitis of various kinds. Examples of non-inflammatory skin conditions encompass, without limitation, non-inflammatory dermatosis, xerosis, pruritus, blistering, etc.

In a preferred embodiment, the inflammatory dermatological condition is psoriasis or a symptom associated therewith.

As used herein, “psoriasis or a symptom associated therewith” is intended to encompass any type or classification of psoriasis and any symptom associated therewith. For example, the term “psoriasis or a symptom associated therewith” includes plaque psoriasis, pustular psoriasis, guttate psoriasis (small, drop like spots) and flexural psoriasis, nail psoriasis, psoriatic arthritis and erythrodermic psoriasis, and their associated symptoms. The term also includes nonpustular psoriasis, such as psoriasis vulgaris (chronic stationary psoriasis, plaque-like psoriasis), psoriatic erythroderma (erythrodermic psoriasis); and pustular psoriasis, such as generalized pustular psoriasis (pustular psoriasis of von Zumbusch) (liquid-filled yellowish small blisters), pustulosis palmaris et plantaris (palmoplantar pustulosis (primarily affecting the palms and the soles), pustular psoriasis of the Barber type, pustular psoriasis of the extremities), annular pustular psoriasis, acrodermatitis continua and impetigo herpetiformis, and their associated symptoms. The term also encompasses drug-induced psoriasis, inverse psoriasis (in the folds like of the underarms, navel, and buttocks), napkin psoriasis, and seborrheic-like psoriasis, and their associated symptoms.

The degree or the severity of the psoriasis may vary. The degree of severity is generally based on the proportion of body surface area affected, disease activity (degree of plaque redness, thickness and scaling), response to previous therapies, and the impact of the disease on the person. The term “psoriasis or a symptom associated therewith” encompasses the mild (affecting less than 3% of the body area), moderate (affecting 3-10% of the body area) or severe (more than 10% of the body area) psoriasis and its symptoms.

Psoriasis typically looks like red or pink areas of thickened, raised, and dry skin. It classically affects areas over the elbows, knees, and scalp. Essentially any body area may be involved. It tends to be more common in areas of trauma, repeat rubbing, use, or abrasions. Psoriasis has many different appearances. It may be small flattened bumps, large thick plaques of raised skin, red patches, and pink mildly dry skin to big flakes of dry skin that flake off. Sometimes pulling of one of these small dry white flakes of skin causes a tiny blood spot on the skin. This is medically referred to as a special diagnostic sign in psoriasis called the Auspitz sign.

Genital lesions, especially on the head of the penis, are common. Psoriasis in moist areas like the navel or area between the buttocks (intergluteal folds) may look like flat red patches. These atypical appearances may be confused with other skin conditions like fungal infections, yeast infections, skin irritation, or bacterial Staph infections.

On the nails, it can look like very small pits (pinpoint depressions or white spots on the nail) or as larger yellowish-brown separations of the nail bed called “oil spots.” Nail psoriasis may be confused with and incorrectly diagnosed as a fungal nail infection.

On the scalp, it may look like severe dandruff with dry flakes and red areas of skin.

In view of the present disclosure, a skin area affected by, or is prone to be affected by, a dermatological condition, such as psoriasis, can be identified using any diagnostic signs or means known in the art, and can be treated by methods according to embodiments of the present invention.

As used herein, “berberine” refers to a compound of formula (I):

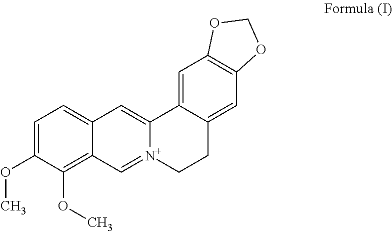 [SMILES: COc1ccc2cc3[n+](cc2c1OC)CCc1cc2c(cc1-3)OCO2]

or an isomer, ester, prodrug, derivative or pharmaceutically acceptable salt thereof. The isomer can be any possible isomeric form of formula (I), such as an optical isomer, enantiomer, diastereomer, racemate or racemic mixture of formula (I). Berberine is strongly yellow colored.

Berberine can be chemically synthesized using methods known to those skilled in the art in view of its structure.

Berberine can also be extracted from a plant, such as Berberis (e.g. Berberis aquifolium, Berberis vulgaris, and Berberis aristata), Mahonia aquifolium, Cortex phellodendri, Hydrastis canadensis (Goldenseal), Phellodendron amurense (Amur Cork Tree, Huang Bai, Huang Po, Po Mu), Coptis chinensis (Chinese Goldthread, Huang-Lian), Tinospora cordifolia, Argemone mexicana (Prickly Poppy), or Eschscholzia californica (Californian Poppy).

As used herein, “indigo naturalis composition” is understood to mean a composition comprising at least one composition or compound selected from the group consisting of an indigo naturalis, an extract of an indigo naturalis, an active agent of an indigo naturalis, and a combination thereof.

By “indigo naturalis,” it is understood that it is a dark-blue plant pigment extracted from an indigo-producing plant such as Baphicacanthus cusia (Nees) Bremek, Polygonum tinctorium Lour, Isatis indigotica Fort, Isatis tinctoria LINN, etc.

The “extract of indigo naturalis” of the present invention includes, but is not limited to, extracts of the dark-blue plant pigment from an indigo-producing plant in a solvent, an oil, or a surfactant solution.

The solvents used for the extract according to the present invention include, but are not limited to, an organic solvent such, as polar organic solvents, non-polar organic solvents, and the mixtures thereof. In a preferred embodiment of this invention, the solvent is a polar solvent. Furthermore, the most preferred solvents of the present invention are THF, dioxane, chloroform, isopropanol, and 2-Butanone.

The oils used for the extract according to the present invention include, but are not limited to, vegetable oils, animal oils, mineral oils, and the mixtures thereof.

The surfactants useful for the extract according to the present invention include, but are not limited to, anionic, cationic, non-ionic and/or amphoteric surfactants, such as acyl amino acids, ethoxylated amines, acyl/dialkyl ethylene diamine, alkanolamides, etc.

The “active agent of an indigo naturalis” of the present invention includes, but is not limited to, indigo, indirubin, tryptanthrin, N-methylisoindigotin (meisoindigo), N-acetyl-indirubin, isoindigo, isatin, or N-Phenyl-2-naphtylamine. The preferred active agents of an indigo naturalis include indigo, indirubin, and tryptanthrin.

In addition to the indigo, indirubin, and tryptanthrin, the extracts of indigo naturalis of the present invention may further comprise a relatively small amount of one or more of the following compounds that are known to be present indigo naturalis. They include, but are not limited to, N-methylisoindigotin (meisoindigo); N-acetyl-indirubin; isoindigo; isatin; and N-phenyl-2-naphtylamine etc. The other organic components from the indigo naturalis can account for 20% to 90% (w/w) of the total amount of all components extracted from the indigo naturalis.

Furthermore, according to the traditional use of the Chinese herbal medicine, it is possible that those components also contributed to the efficacy of the extracts, either due to the efficacy of their own independently, or due their synergetic effects with other components in the extracts, but they may or may not have any efficacy when administered alone.

The indigo naturslis composition can be extracted from a plant. The active agent of an indigo naturalis can be isolated from a plant extract or chemically synthesized using methods known to those skilled in the art in view of their structures.

It is surprisingly discovered in the present invention that the combination of an effective amount of berberine with an effective amount of indigo naturslis composition achieves synergistic effect. This allows the design of novel compositions with smaller amounts of the active ingredients than that used in the prior art for effective treatment of a dermatological condition while reducing or avoiding the adverse effects. The combination composition according to an embodiment of the present invention has an aesthetically pleasing color for topical application, and provides more effective treatment of the dermatological condition than the additive effect of the active ingredients.

In view of the present disclosure, standard in vivo or in vitro procedures can be conducted to determine the effect of each of the active ingredients alone or in combination in a composition according to embodiments of the present invention. For example, the effect of the composition can be evaluated using reconstructed human epidermis (RHE).

The topical composition of this invention may be in the form of suspensions of polymeric or lipid vesicles or nanospheres or microspheres or polymeric patches and hydrogels allowing controlled release. These topical-route compositions may be either in anhydrous form or in aqueous form depending on the clinical indication.

The suitable dosage form the topical composition of this invention includes, but is not limited to, solutions, aerosols and non-aerosol sprays, shaving creams, powders, mousses, lotions, gels, sticks, ointments, pastes, creams, shampoos, shower, gel, body washes or face washes.

The topical composition according to this invention can additionally comprise a topically acceptable carrier or pharmaceutically acceptable carrier widely employed in the art of drug-manufacturing. For instance, the carrier may include one or more of the following agents: solvents, emulsifiers, suspending agents, decomposers, binding agents, excipients, stabilizing agents, chelating agents, diluents, gelling agents, preservatives, lubricants, absorption delaying agents, liposomes, and the like.

Preferably, the topical composition according to this invention is formulated into an external preparation that can be directly applied to a psoriatic lesion. The external preparation suitable for the pharmaceutical composition according to this invention may be an emulsion, a gel, an ointment, a cream, a patch, an embrocation, an aerosol, a spray, a lotion, a serum, a paste, a foam, or a drop. In a preferred embodiment of this invention, the topical composition is formulated into an external preparation by admixing the extracts of indigo naturalis according to this invention with a base that is well known and commonly used in the art.

For example, the base suitable for producing the external preparation according to this invention may include one or more of the following additives: hydrocarbons (e.g., petroleum jelly and white petrolatum), wax (e.g., paraffin and yellow wax), preserving agents, antioxidant, surfactants, absorption enhancers, stabilizing agents, gelling agents, active agents, odor absorbers, or fragrances. The choice and amount of these additives are within the expertise of those skilled in the art.

In a preferred embodiment of this invention, the topical composition is formulated into an ointment. In another preferred embodiment of this invention, the topical composition is formulated into an oil drop.

The present invention also relates to a method of treating a dermatological condition, such as psoriasis. The method comprises topically administering to the skin of the subject in need of the treatment a therapeutically effective amounts of a topical composition comprising: a) berberine; b) at least one indigo naturalis composition; and c) a topically acceptable carrier, wherein a synergistic effect is obtained between the berberine and the at least one indigo naturalis composition.

According to the present invention, the topical compositions of the invention are intended for treating the skin, the nails, the scalp, and mucous membranes.

One skilled in the art will recognize that the therapeutically effective amount of the topical composition to be used in the instant invention can vary with factors, such as the particular subject, e.g., age, diet, health, etc., severity and complications and types of the skin condition sought to be treated or prevented, the formulation used, etc. Dosages and dosing frequency will be determined by a trained medical professional depending on the activity of the compounds used, the characteristics of the particular topical formulation, and the identity and severity of the dermatologic disorder treated or prevented.

To treat or prevent a skin condition, such as psoriasis or a symptom associated therewith, in view of the present disclosure, the topically administrable compositions of the invention can be topically applied directly to the affected area in any conventional manner known in the art, e.g., by dropper or applicator stick, as a mist via an aerosol applicator, via an intradermal or transdermal patch, or by simply spreading a formulation of the invention onto the affected area with fingers.

Methods of the present invention can be used in conjunction with one or more other treatments and medications for the skin condition.

The other medicament or treatment can be administered to the subject simultaneously with, or in a sequence and within a time interval of the administration of the topical composition, such that the active ingredients or agents can act together to treat or prevent psoriasis and symptoms associated therewith. For example, the other medicament or treatment can be administered in the same or separate formulations at the same or different times.

Any suitable route of administration can be employed to deliver the additional treatment or medication including, but not limited to, oral, intraoral, rectal, parenteral, topical, epicutaneous, transdermal, subcutaneous, intramuscular, intranasal, sublingual, buccal, intradural, intraocular, intrarespiratory, or nasal inhalation.

This invention will be better understood by reference to the non-limiting examples that follow, but those skilled in the art will readily appreciate that the examples are only illustrative of the invention as described more fully in the claims which follow thereafter.

### Example 1

### Evaluation of a Topical Composition Using In Vitro Reconstructed Human Epidermis

In vitro reconstructed human epidermis (RHE) from SkinEthic Laboratories was used to evaluate the anti-inflammatory effect of topical compositions. The RHE consists of normal human keratinocytes cultured on an inert polycarbonate filter at the air-liquid interface in a chemically defined medium. Using the RHE model, inflammatory process can be monitored by gene expression profiling of selected genes, e.g., via TaqMan Low-Density Array (TLDA) technology, and cytokine expression profiling of selected proteins, e.g., via luminex.

The RHE model was validated with known treatments. It was found that, stimulus, such as tetradecanoylphorbol myristate acetate and lipopolysaccharide (TPA+LPS), TNFα, IFNγ or IL22, induced inflammatory responses, activated gene expression of selected genes and increased cytokine expression of selected proteins. Dermoval, an existing treatment of psoriasis, inhibited inflammation induced by TPA+LPS, TNFα, IFNγ or IL22, as shown by gene expression as well as cytokine expression analysis from RHE.

An ointment comprising a combination of berberine and an indigo naturalis composition was prepared. The berberine and the indigo naturalis composition was each extracted from plants. The anti-inflammatory effect of the ointment was studied using the RHE model. It was found that the ointment significantly inhibited inflammatory responses as shown by inhibition of gene expression induced by TPA+LPS or IL-22, and inhibition of cytokine production induced by TPA+LPS, TNFα or IFNγ. The ointment has a profile of cytokine production similar to a TNF inhibitor. Results from the RHE study indicated that a combination of berberine and indigo naturalis clearly displayed anti-inflammatory responses indicative of its effectiveness in treating inflammatory skin disorders, such as psoriasis.

Compositions containing individual active ingredients and combinations of the active ingredients at various ratios and amounts are studied using the RHE model. Synergetic fixed dose combination of berberine and indigo naturalis for the treatment of psoriasis is then formulated and further tested in RHE and other studies, e.g., in animal model and clinical trials.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/We claim:

1. A topical composition for treating an inflammatory dermatological condition, comprising:
a) berberine
b) at least one indigo naturalis composition; and
c) a topically acceptable carrier,
wherein a synergistic effect is obtained between the berberine and the at least one indigo naturalis composition.

2. The topical composition according to claim 1, wherein the indigo naturalis composition comprises at least one composition or compound selected from the group consisting of an indigo naturalis, an extract of indigo naturalis, an active agent of an indigo naturalis, and the combination thereof.

3. The topical composition according to claim 2, wherein the active agent of an indigo naturalis is indigo, indirubin, tryptanthrin, N-methylisoindigotin (meisoindigo), N-acetyl-indirubin, isoindigo, isatin, or N-phenyl-2-naphtylamine.

4. The topical composition according to claim 3, wherein the active agent of an indigo naturalis is indigo, indirubin or tryptanthrin.

5. The topical composition according to claim 1, wherein the beberine is substantially isolated.

6. The topical composition according to claim 1, wherein the topical composition is an ointment, a cream, a milk, a pomades, a powder, an impregnated pad, a solution, a gel, a spray, a lotion, a suspension, an oil, a solid stick, an aerosol, a foam or a mousse.

7. The topical composition according to claim 1, wherein the inflammatory dermatological condition is psoriasis.

8. The topical composition according to claim 1, wherein the psoriasis is psoriatic skin lesions or psoriatic nail lesions.

9. The topical composition according to claim 1, wherein the psoriasis is selected from chronic plaque psoriasis, guttate psoriasis, erythrodermic psoriasis, pustular psoriasis, and the combinations thereof.

10. A method of treating an inflammatory dermatological condition in a subject comprising topically administering to the skin of the subject in need of the treatment a therapeutically effective amount of a topical composition comprising:
a) berberine
b) at least one indigo naturalis composition; and
c) a topically acceptable carrier,
wherein a synergistic effect is obtained between the berberine and the at least one indigo naturalis composition.

11. The method according to claim 10, wherein the indigo naturalis composition comprises at least one composition or compound selected from the group consisting of an indigo naturalis, an extract of indigo naturalis, an active agent of an indigo naturalis, and a combination thereof.

12. The method according to claim 10, wherein the active agent of an indigo naturalis is indigo, indirubin, tryptanthrin, N-methylisoindigotin (meisoindigo), N-acetyl-indirubin, isoindigo, isatin, or N-phenyl-2-naphtylamine.

13. The method according to claim 12, wherein the active agent of an indigo naturalis is indigo, indirubin, or tryptanthrin.

14. The method according to claim 13, wherein the beberine is substantially isolated.

15. The method according to claim 10, wherein the inflammatory dermatological condition is psoriasis.

16. The method according to claim 10, wherein the psoriasis is psoriatic skin lesions or psoriatic nail lesions.

17. The method according to claim 10, wherein the psoriasis is selected from chronic plaque psoriasis, guttate psoriasis, erythrodermic psoriasis, pustular psoriasis, and the combinations thereof.
